(19) United States
(12) Patent Application Publication
(10) Pub. No.: US 2026/0240725 A1
(43) Pub. Date: Aug. 20, 2026

(54) APPARATUS, SYSTEM, AND METHOD FOR STORING MEDICAL, PHARMACEUTICAL, OR BIOLOGICAL MEDIA

(71) Applicant: SAINT-GOBAIN PERFORMANCE PLASTICS CORPORATION, Malvern, PA (US)

(72) Inventors: James David BOGHOSIAN, Natick, MA (US); Katie L. CAMPBELL, Northborough, MA (US); Nicolas DROLET, Chelmsford, MA (US); Jonathon W. WHEATLEY, Traverse City, MI (US); Elizabeth Grace TOWLE, Worcester, MA (US)

(21) Appl. No.: 19/545,524

(22) Filed: Feb. 20, 2026

Related U.S. Application Data
(60) Provisional application No. 63/760,968, filed on Feb. 20, 2025.

(51) Int. Cl. A61J 1/16; A61J 1/10

(52) U.S. Cl. CPC A61J 1/16; A61J 1/10

(57) ABSTRACT

An apparatus including: a core including an annular body defining an internal void oriented down a central axis having a top axial surface and a bottom axial surface; at least one retainer including a body adapted to couple to the top axial surface or the bottom axial surface of the core; and at least one flexible film membrane bonded to the at least one retainer, where the at least one flexible film membrane at least partially encloses the internal void to provide for a medically, biologically, or pharmaceutically active environment within the internal void of the core, where the at least one retainer includes a rigid inner portion including a plurality of through holes, where the at least one retainer is adapted to hold the at least one flexible film membrane in a substantially static axial position.

### CROSS-REFERENCE TO RELATED APPLICATION(S)

This Application claims priority under 35 U.S.C. § 119(e) to U.S. Provisional Patent Application No. 63/760,968 entitled “APPARATUS, SYSTEM, AND METHOD FOR STORING MEDICAL, PHARMACEUTICAL, OR BIOLOGICAL MEDIA,” filed February 20, 2025, by James David BOGHOSIAN et al., which is assigned to the current assignee hereof and is incorporated herein by reference in its entirety.

### FIELD OF THE DISCLOSURE

The present disclosure relates to apparatuses, systems, and methods for storing medical, pharmaceutical, or biological media.

### RELATED ART

Many pharmaceutical and bioprocessing processes utilize storage containers for storing fluid associated with medical, pharmaceutical, or biological media such as, but not limited to, cell cultures. In certain instances, the container can include a bag having at least one flexible portion – such as a flexible sidewall. These flexible sidewalls may be challenging to manufacture and are prone to wrinkling, preventing efficient storage and lessening the desired modification or sustainment of the medical, pharmaceutical, or biological media within the container. Further, these flexible sidewalls may at least partially serve as membranes for gas diffusion into the container, which may also be lessened in efficiency by wrinkling. Further, these flexible sidewalls may bow or displace inwardly when the fluid is emptied from the container, increasing the likelihood of flexible sidewall failure with repeated use of the container. Therefore, the medical, pharmaceutical, and bioprocessing industries continue to demand better containers and associated technologies to permit more efficient storage and operation involving modification or sustainment of medical, pharmaceutical, and biological media.

### BRIEF DESCRIPTION OF THE DRAWINGS

Embodiments are illustrated by way of example and are not intended to be limited in the accompanying figures.

FIG. 1A includes a side perspective view of an apparatus in accordance with an embodiment.

FIG. 1B includes a side perspective view of an apparatus in accordance with an embodiment.

FIG. 1C includes a cross-sectional view of an apparatus in accordance with an embodiment.

FIG. 1D includes a close-up cross-sectional view of an apparatus as viewed within circle D of FIG. 1C in accordance with an embodiment.

FIG. 1E1 includes a top view of a core for an apparatus in accordance with an embodiment.

FIG. 1E2 includes a side view of a core for an apparatus in accordance with an embodiment.

FIG. 1E3 includes an isometric view of a core for an apparatus in accordance with an embodiment.

FIG. 1F1 includes a top view of a retainer for an apparatus in accordance with an embodiment.

FIG. 1F2 includes a side view of a retainer for an apparatus in accordance with an embodiment.

FIG. 1F3 includes an isometric view of a retainer for an apparatus in accordance with an embodiment.

FIG. 1G1 includes a top view of a membrane for an apparatus in accordance with an embodiment.

FIG. 1G2 includes an isometric view of a membrane for an apparatus in accordance with an embodiment.

FIG. 1H1 includes an isometric view of a retainer for an apparatus in accordance with an embodiment.

FIG. 1H2 includes an isometric of a retainer and a membrane for an apparatus in accordance with an embodiment.

FIG. 1H3 includes an isometric cross-sectional view of a retainer and a membrane for an apparatus in accordance with an embodiment.

FIG. 1H4 includes an isometric cross-sectional view of a membrane for an apparatus in accordance with an embodiment.

### DETAILED DESCRIPTION

The following description in combination with the figures is provided to assist in understanding the teachings disclosed herein. The following discussion will focus on specific implementations and embodiments of the teachings. This focus is provided to assist in describing the teachings and should not be interpreted as a limitation on the scope or applicability of the teachings. However, other embodiments can be used based on the teachings as disclosed in this application.

The terms “comprises,” “comprising,” “includes,” “including,” “has,” “having” or any other variation thereof, are intended to cover a non-exclusive inclusion. For example, a method, article, or apparatus that comprises a list of features is not necessarily limited only to those features but may include other features not expressly listed or inherent to such method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” is employed to describe elements and components described herein. This is done merely for convenience and to give a general sense of the scope of the invention. This description should be read to include one, at least one, or the singular as also including the plural, or vice versa, unless it is clear that it is meant otherwise. For example, when a single item is described herein, more than one item may be used in place of a single item. Similarly, where more than one item is described herein, a single item may be substituted for that more than one item.

Unless otherwise defined, all technical and scientific terms used herein have the same meaning as commonly understood by one of ordinary skill in the art to which this invention belongs. The materials, methods, and examples are illustrative only and not intended to be limiting. To the extent not described herein, many details regarding specific materials and processing acts are conventional and may be found in textbooks and other sources within the medical, pharmaceutical, and biological media processing and storage arts.

In accordance with one or more of the embodiments described herein, an apparatus may generally include: a core including an annular body defining an internal void oriented down a central axis having a top axial surface and a bottom axial surface; at least one retainer including an annular body adapted to couple to the top axial surface or the bottom axial surface of the core to form a circumferential engagement cavity defined radially between the at least one retainer and the core; at least one ring seal disposed within the engagement cavity; and at least one flexible film membrane partially disposed within the engagement cavity, where the at least one flexible film membrane at least partially encloses the internal void to provide for a medically, biologically, or pharmaceutically active environment within the internal void of the core.

In accordance with one or more of the embodiments described herein, an apparatus may generally include: a core including an annular body defining an internal void oriented down a central axis having a top axial surface and a bottom axial surface; at least one retainer including a body adapted to couple to the top axial surface or the bottom axial surface of the core; and at least one flexible film membrane bonded to the at least one retainer, where the at least one flexible film membrane at least partially encloses the internal void to provide for a medically, biologically, or pharmaceutically active environment within the internal void of the core, where the at least one retainer includes a rigid inner portion including a plurality of through holes, where the at least one retainer is adapted to hold the at least one flexible film membrane in a substantially static axial position.

In accordance with one or more of the embodiments described herein, a system may generally include: a medium including a medical, pharmaceutical, or biological component; and an apparatus including: a core including an annular body defining an internal void oriented down a central axis having a top axial surface and a bottom axial surface, at least one retainer including a body adapted to couple to the top axial surface or the bottom axial surface of the core, and at least one flexible film membrane bonded to the at least one retainer, where the at least one flexible film membrane at least partially encloses the internal void about the medium to provide for a medically, biologically, or pharmaceutically active environment within the internal void of the core, where the at least one retainer includes a rigid inner portion including a plurality of through holes, where the at least one retainer is adapted to hold the at least one flexible film membrane in a substantially static axial position.

In accordance with one or more of the embodiments described herein, a method may generally include: providing a medium including a medical, pharmaceutical, or biological component; providing an apparatus including: a core including an annular body defining an internal void oriented down a central axis having a top axial surface and a bottom axial surface, at least one retainer including an annular body adapted to couple to the top axial surface or the bottom axial surface of the core, where the at least one retainer includes a rigid inner portion including a plurality of through holes, and at least one flexible film membrane bonded to the at least one retainer, where the at least one flexible film membrane at least partially encloses the internal void; disposing the medium within the internal void to provide for a medically, biologically, or pharmaceutically active environment within the internal void of the core; and removing the medium from the internal void while maintaining the at least one film membrane in a substantially static axial position via the at least one retainer.

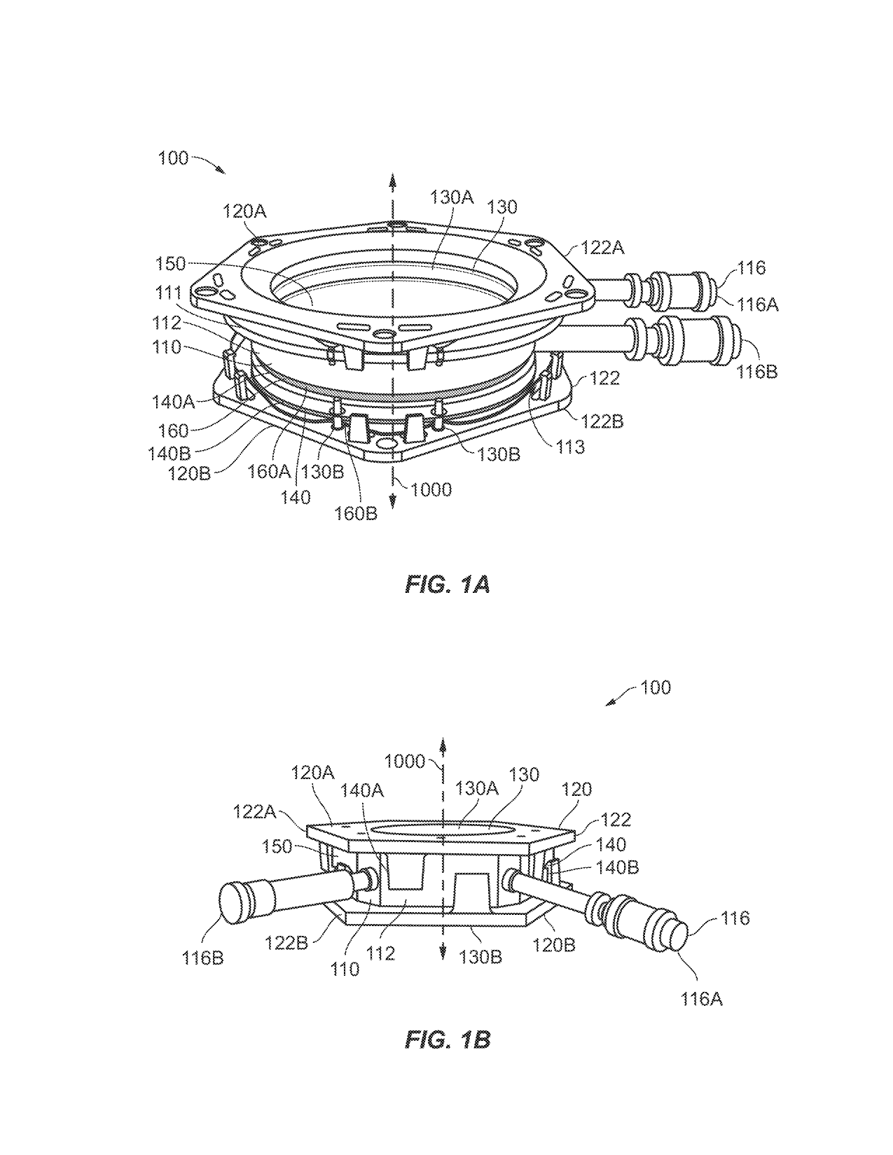

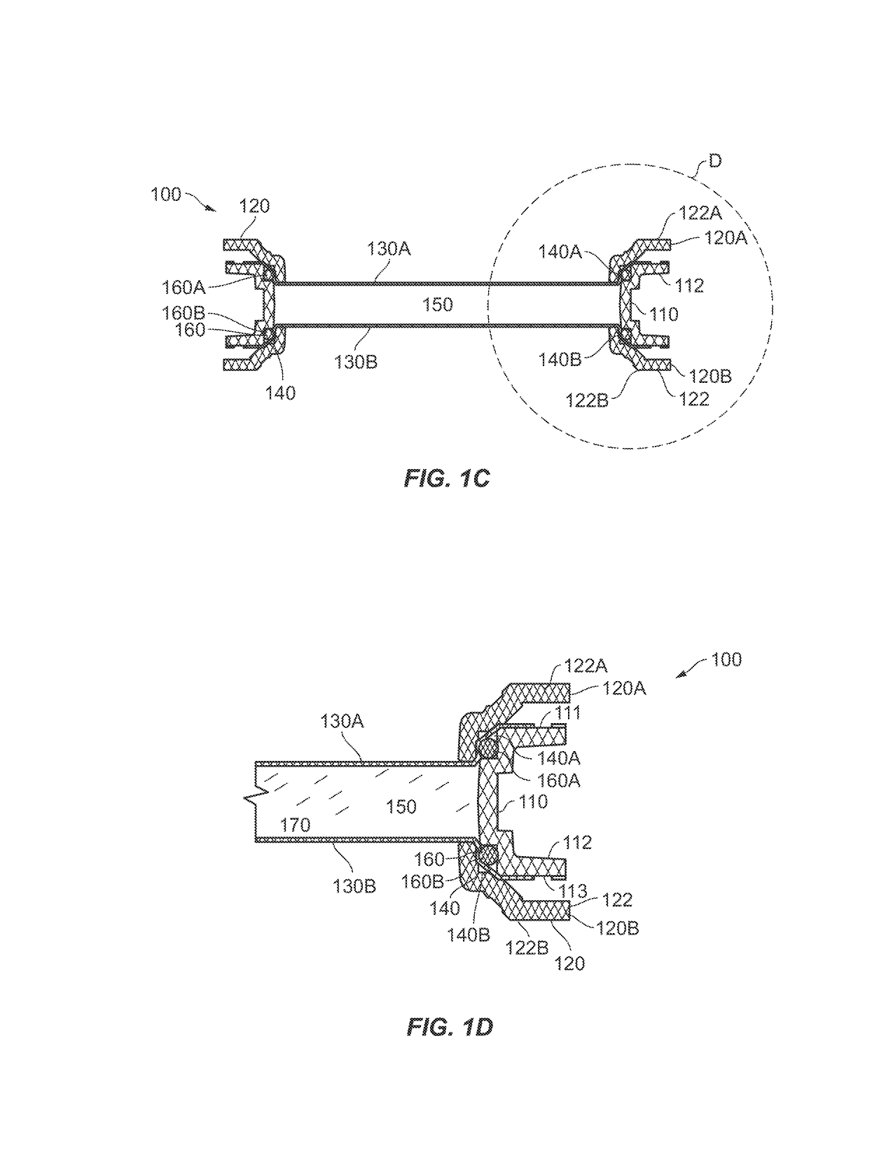

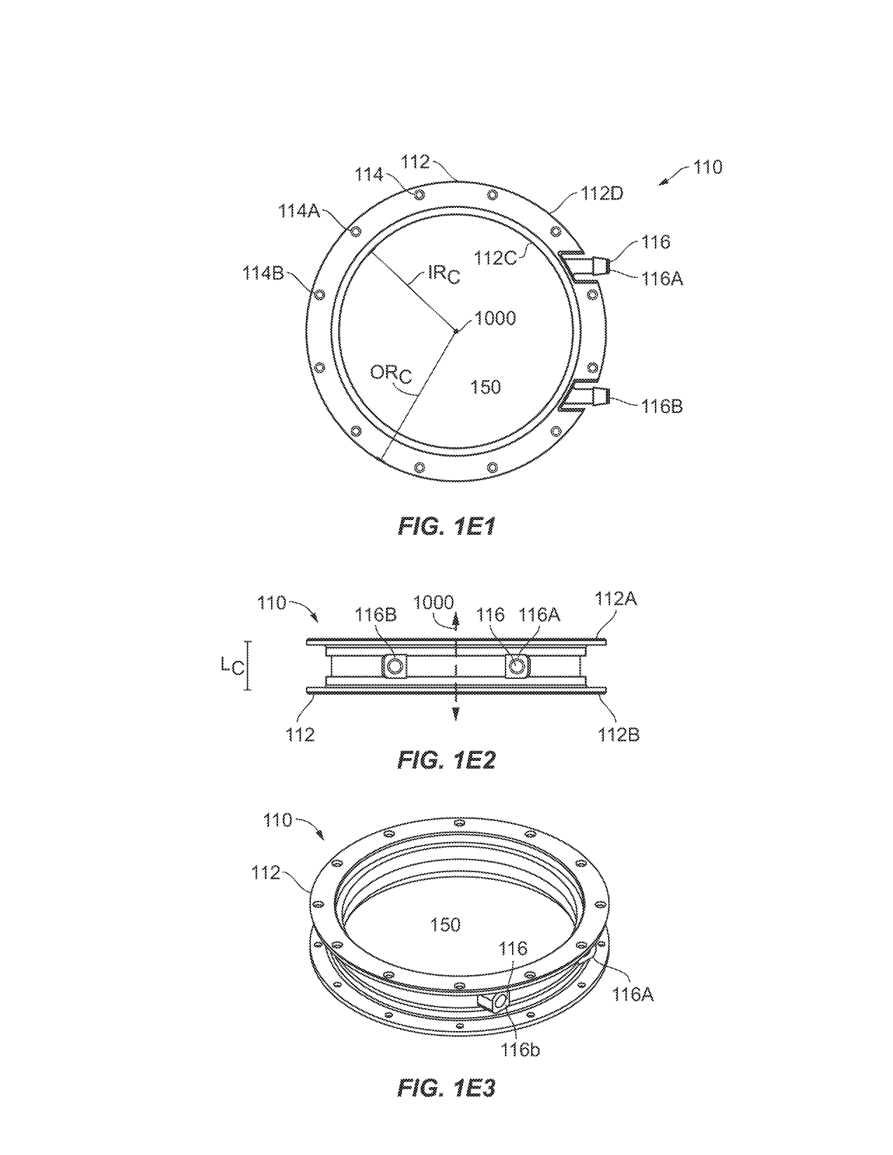

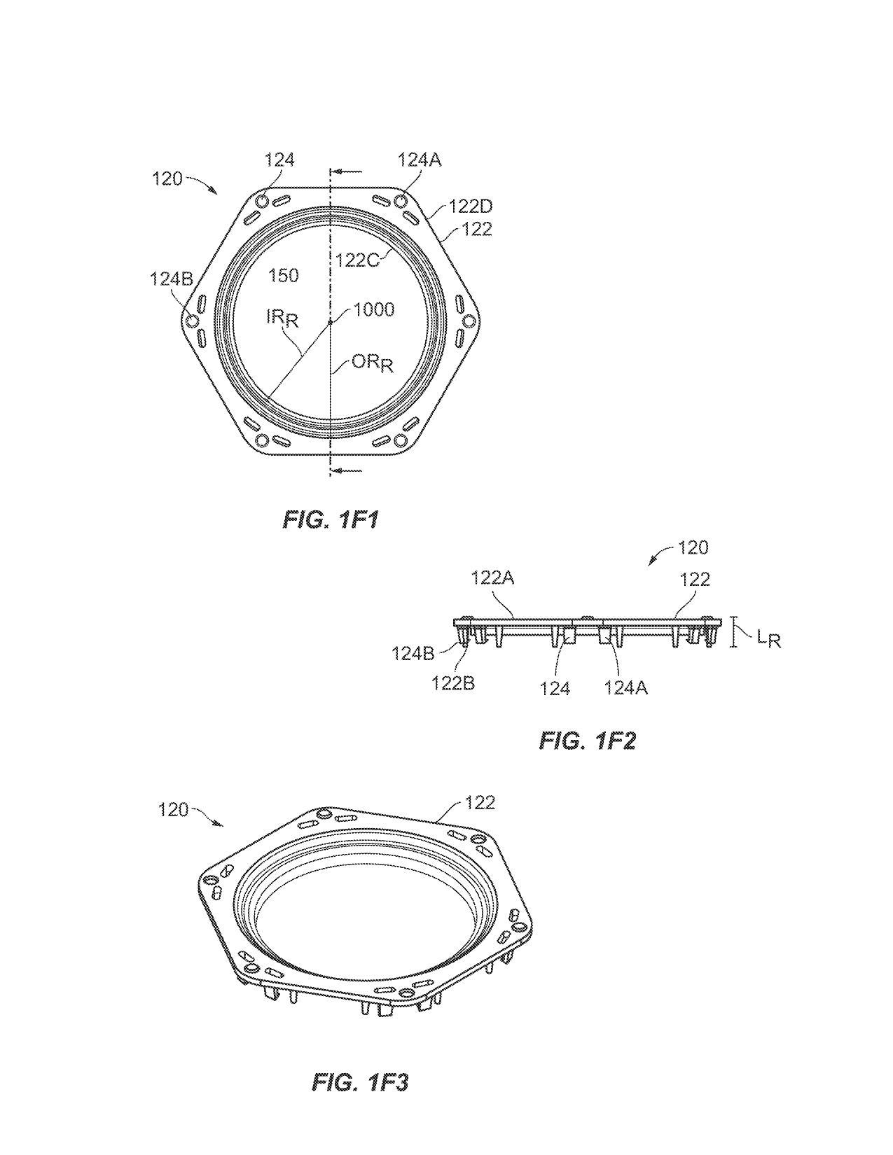

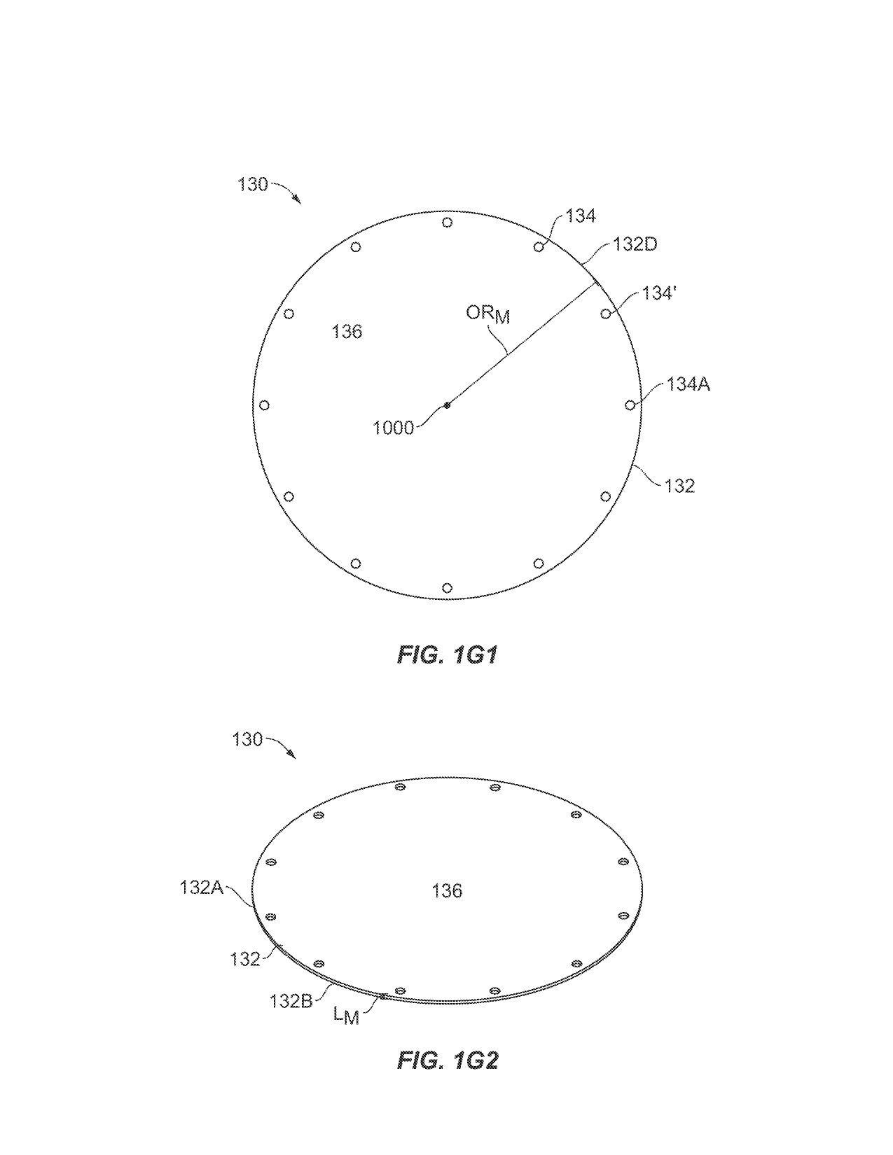

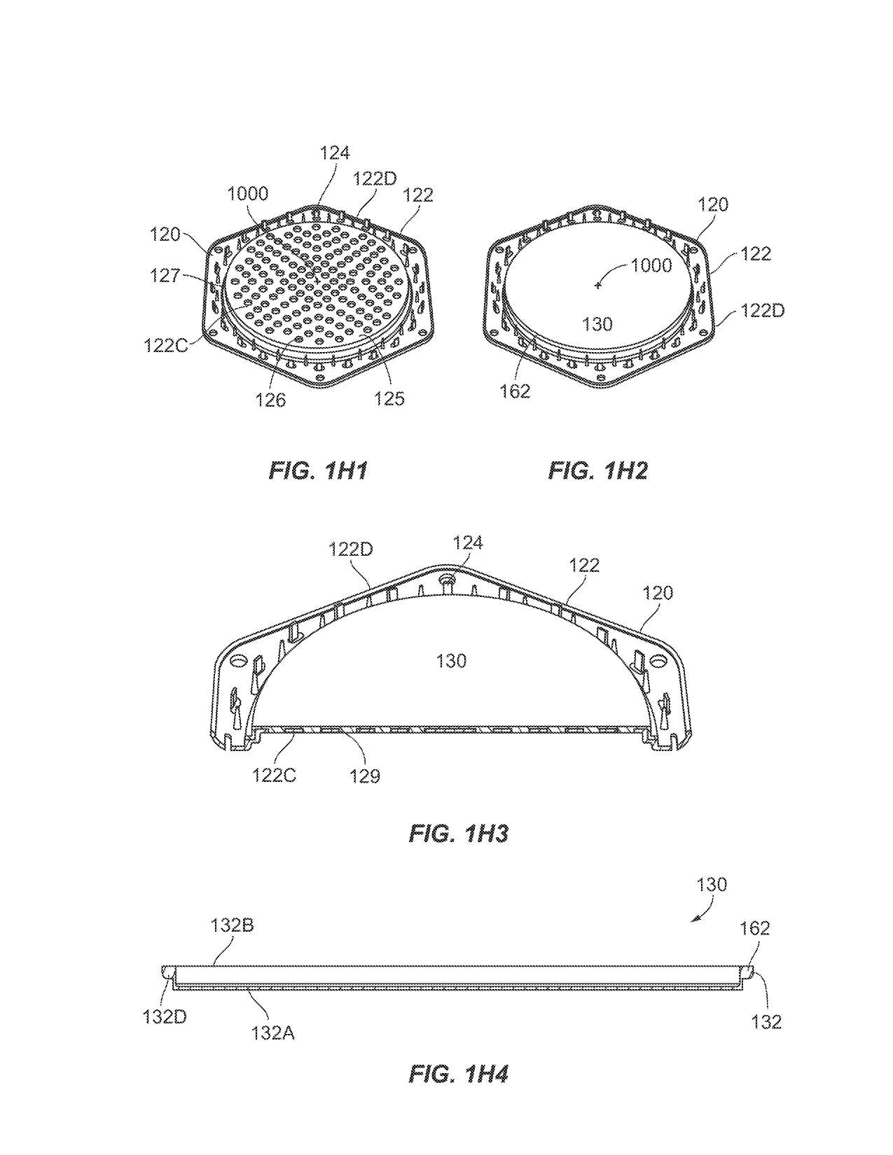

FIG. 1A includes a side perspective view of an apparatus 100 in accordance with an embodiment. FIG. 1B includes a side perspective view of an apparatus 100 in accordance with an embodiment. FIG. 1C includes a cross-sectional view of an apparatus in accordance with an embodiment. FIG. 1D includes a close-up cross-sectional view of an apparatus as viewed within circle D of FIG. 1C in accordance with an embodiment. It is contemplated that reference numerals are corresponding across all Figures disclosed herein. As shown in FIGS. 1A-1D, the apparatus 100 can generally include a core 110 oriented down a central axis 1000. In a number of embodiments, the core 110 may include an annular body 112. As used herein, the term “annular” may be defined as having a solid structure oriented 360˚ about the central axis 1000. The term “annular” including any cross-sectional shape (e.g. polygonal, oval, circular, semi-circular, or substantially circular cross-section) is contemplated herein. The annular body 112 of the core 110 may define an internal void 150 within an aperture of the annular body 112 down the central axis 1000. Further, as shown best in FIG. 1D, the annular body 112 of the core 110 may define a top axial surface 111 and a bottom axial surface 113 down the central axis 1000.

Further, still referring to FIGS. 1A-1D, the apparatus 100 can generally include at least one retainer 120 oriented down a central axis 1000. In a number of embodiments, as shown, the at least one retainer 120 may be adapted to couple to the top axial surface 111 or the bottom axial surface 113 of the annular body 112 of the core 110 down the central axis 1000. In a number of embodiments, the at least one retainer 120 may include an annular body 122. As shown in FIGS. 1A-1D, the apparatus 100 can generally include a plurality of retainers 120A, 120B including a plurality of annular bodies 122A, 122B. In a number of embodiments, the plurality of retainers 120A, 120B may be adapted to couple to the top axial surface 111 and the bottom axial surface 113 of the annular body 112 of the core 110 down the central axis 1000 (one on each axial side respectively). As shown best in FIGS. 1C-1D, the at least one retainer 120 may be adapted to couple to the top axial surface 111 or the bottom axial surface 113 of the annular body 112 of the core 110 to form a circumferential engagement cavity 140 around a perimeter of the core 100. In a number of embodiments, as shown best in FIGS. 1C-1D, the at least one circumferential engagement cavity 140 may be formed and defined radially between the at least one retainer 120 and the core 110 such that the core 110 forms the outer diameter of the at least one circumferential engagement cavity 140 while the at least one retainer 120 forms the inner diameter of the at least one circumferential engagement cavity 140 relative to the central axis 1000. In a number of embodiments, as shown, the plurality of retainers 120A, 120B may be adapted to couple to the top axial surface 111 and the bottom axial surface 113 of the annular body 112 of the core 110 to form a plurality of circumferential engagement cavities 140A, 140B around a perimeter of the core 100.

Further, as shown best in FIGS. 1A and 1C-1D, the apparatus can generally include at least one ring seal 160 disposed within the engagement cavity 140. As shown in FIGS. 1A and 1C-1D, the apparatus 100 can generally include a plurality of ring seals 160A, 160B, one disposed in each engagement cavity 140A, 140B. The at least one ring seal 160 may have a polygonal, circular, elliptical, cross-sectional shape or may be another type. In a number of embodiments, the at least one ring seal 160 may include an elastomer material.

Further, the apparatus 100 can generally include at least one membrane 130 oriented down a central axis 1000 and at least partially disposed within the engagement cavity 140. As shown in FIGS. 1A-1D, the apparatus 100 can generally include a plurality of membranes 130A, 130B, one at least partially disposed in each engagement cavity 140A, 140B. Further, as shown in FIGS. 1A-1D the at least one membrane 130 may at least partially enclose the internal void 150 to provide for a medically, biologically, or pharmaceutically active environment within the internal void 150 of the core 110 and subsequently the apparatus 100. Further, as shown in FIGS. 1A-1D, the plurality of membranes 130A, 130B may together at least partially enclose the internal void 150 completely in the axial direction down the central axis 1000 to provide for a medically, biologically, or pharmaceutically active environment within the internal void 150 of the core 110 and subsequently the apparatus 100.

FIG. 1E1 includes a top view of a core for an apparatus in accordance with an embodiment. FIG. 1E2 includes a side view of a core for an apparatus in accordance with an embodiment. FIG. 1E3 includes an isometric view of a core for an apparatus in accordance with an embodiment. As shown in FIGS. 1E1-1E3, the core 110 may include an annular body 122 forming an “annular shape” surrounding a bore or aperture defining an internal void 150 down a central axis 1000. In particular embodiments, the core 110 may form a substantially circular shape, as shown. The body 112 of the core 110 may have a first axial end 112A, a second axial end 112B, an inner radial end 112C, and an outer radial end 112D. As shown best in FIG. 1E2, in a number of embodiments, the core 100 may include a radial flange at at least one of the first axial end 112A or the second axial end 112B. As shown, in a number of embodiments, the core 100 may include a radial flange at both of the first axial end 112A and the second axial end 112B forming a substantially U-shaped cross-sectional profile in a plane parallel to the central axis 1000.

In a number of embodiments, as shown best in FIG. 1E1, the core 110 may have an outer radius OR_{C}. For purposes of embodiments described herein, the core 110 may have an outer radius OR_{C} that is the distance from the central axis 1000 to the outer radial end 112D. According to certain embodiments, core 110 may have an outer radius OR_{C} that may be at least about 10 mm, at least 25 mm, at least 50 mm, at least 100 mm, at least about 150 mm, or at least about 200 mm, or at least about 250 mm, or at least about 300 mm, or even at least about 500 mm. According to still other embodiments, core 110 may have an outer radius OR_{C} that may be not greater than about 1500 mm, not greater than about 1200 mm, or even not greater than about 1000 mm. It will be appreciated that the core 110 may have an outer radius OR_{C} that may be within a range between any of the minimum and maximum values noted above. It will be further appreciated that the core 110 may have an outer radius OR_{C} that may be any value between any of the minimum and maximum values noted above.

In a number of embodiments, as shown best in FIG. 1E1, the core 110 may have an inner radius IR_{C}. For purposes of embodiments described herein, the core 110 may have an inner radius IR_{C} that is the distance from the central axis 1000 to the inner radial end 112C. According to certain embodiments, core 110 may have an inner radius IR_{C} that may be at least about 10 mm, at least 25 mm, at least 50 mm, at least 100 mm, at least about 150 mm, or at least about 200 mm, or at least about 250 mm, or at least about 300 mm, or even at least about 500 mm. According to still other embodiments, core 110 may have an inner radius IR_{C} that may be not greater than about 1500 mm, not greater than about 1200 mm, or even not greater than about 1000 mm. It will be appreciated that the core 110 may have an inner radius IR_{C} that may be within a range between any of the minimum and maximum values noted above. It will be further appreciated that the core 110 may have an inner radius IR_{C} that may be any value between any of the minimum and maximum values noted above.

In a number of embodiments, as shown best in FIG. 1E2, the core 110 can have an axial length (or thickness) L_{C}. For purposes of embodiments described herein, the length L_{C} of the core 110 is the distance from the first axial end 112A to the second axial end 112B. According to certain embodiments, the length L_{C} of the core 110 may be at least about 5 mm, at least about 10 mm, at least 25 mm, at least 50 mm, at least 100 mm, at least about 150 mm, or at least about 200 mm, or at least about 250 mm, or at least about 300 mm, or even at least about 500 mm. According to still other embodiments, the length L_{C} of the core 110 may be not greater than about 1500 mm, not greater than about 1200 mm, or even not greater than about 1000 mm. It will be appreciated that the length L_{C} of the core 110 may be within a range between any of the minimum and maximum values noted above. It will be further appreciated that the length L_{C} of the core 110 may be any value between any of the minimum and maximum values noted above.

In a number of embodiments, as shown best in FIG. 1E1, the core 110 may include at least one axially oriented coupling component 114. In a number of embodiments, the at least one axially oriented coupling component 114 of the core 110 may couple to a neighboring retainer 120 within the apparatus. In a number of embodiments, as shown best in FIG. 1E1, the core 110 may include a plurality of axially oriented coupling components 114A, 114B. In a number of embodiments, the at least one axially oriented coupling component 114 may include at least one of nuts, bolts, bearings, battens, buckles, clips, flanges, frogs, grommets, hook-and-eyes, latches, pegs, nails, rivets, tongue and grooves, screw anchors, snap fasteners, stitches, threaded fasteners, ties, toggle bolts, wedges anchors, screws, bolts, clamps, clasps, clips, latches, pins, rivets, ties, nails, bores, welds, or combinations thereof. In particular embodiments, as shown in FIG. 1E1, the at least one axially oriented coupling component 114 may include a bore or groove.

In a number of embodiments, as shown best in FIGS. 1E1-1E3, the core 110 may include at least one access port 116 allowing for entrance or exit of a medium into or out of the apparatus 100. In a number of embodiments, as shown best in FIGS. 1E1-1E3, the core 110 may include at least a plurality of access ports 116A, 116B allowing for entrance or exit of a medium into or out of the apparatus 100. In particular embodiments, as shown in FIGS. 1E1-1E3, the at least one access port 116 may include a bore. In a number of embodiments, the at least one access port 116 may be adapted to permit, for example, sampling, transport, draining, or removal of medium from the apparatus 100. In a particular embodiment, at least one of the one or more access ports 116 can include at least one fluid port 11, at least two access ports 116, at least three access ports 116, at least four access ports 116, or at least five access ports 116. In another embodiment, the apparatus 100 can include no greater than twenty access ports 116, no greater than fifteen access ports 116, or no greater than ten access ports 116. In the illustrated embodiment, the access ports 116 may have a same shape and size. In other embodiments, the access ports 116 can have different shapes, sizes, lumen diameters, operational capacities, or any combination thereof relative to one another. In an embodiment, the one or more access ports 116 can be coupled with fluid lines adapted to permit transport of medium from the apparatus 100 to an equipment or other medium destination.

FIG. 1F1 includes a top view of a retainer for an apparatus in accordance with an embodiment. FIG. 1F2 includes a side view of a retainer for an apparatus in accordance with an embodiment. FIG. 1F3 includes an isometric view of a retainer for an apparatus in accordance with an embodiment. As shown in FIGS. 1F1-1F3, the at least one retainer 120 may include an annular body 122 forming an “annular shape” surrounding a bore or aperture defining an internal void 150 corresponding to the internal void of the apparatus 100 down a central axis 1000. In particular embodiments, the at least one retainer 120 may form a substantially hexagonal shape, as shown. The body 122 of the at least one retainer 120 may have a first axial end 122A, a second axial end 122B, an inner radial end 122C, and an outer radial end 122D. As shown best in FIG. 1F2, in a number of embodiments, the at least one retainer 120 may include a radial flange at at least one of the first axial end 112A or the second axial end 112B. As shown, in a number of embodiments, the at least one retainer 120 may include a radial flange at both of the first axial end 122A and the second axial end 122B (the radial flange at the second end being an axially oriented coupling component as explained below) forming a substantially L-shaped cross-sectional profile in a plane parallel to the central axis 1000.

In a number of embodiments, as shown best in FIG. 1F1, the at least one retainer 120 may have an outer radius OR_{R}. For purposes of embodiments described herein, the at least one retainer 120 may have an outer radius OR_{R} that is the distance from the central axis 1000 to the outer radial end 122D. According to certain embodiment, the at least one retainer 120 may have an outer radius OR_{R} that may be at least about 10 mm, at least 25 mm, at least 50 mm, at least 100 mm, at least about 150 mm, or at least about 200 mm, or at least about 250 mm, or at least about 300 mm, or even at least about 500 mm. According to still other embodiments, the at least one retainer 120 may have an outer radius OR_{R} that may be not greater than about 1500 mm, not greater than about 1200 mm, or even not greater than about 1000 mm. It will be appreciated that the at least one retainer 120 may have an outer radius OR_{R} that may be within a range between any of the minimum and maximum values noted above. It will be further appreciated that the at least one retainer 120 may have an outer radius OR_{R} that may be any value between any of the minimum and maximum values noted above.

In a number of embodiments, as shown best in FIG. 1F1, the at least one retainer 120 may have an inner radius IR_{R}. For purposes of embodiments described herein, the at least one retainer 120 may have an inner radius IR_{R} that is the distance from the central axis 1000 to the inner radial end 122C. According to certain embodiment, the at least one retainer 120 may have an inner radius IR_{R} that may be at least about 10 mm, at least 25 mm, at least 50 mm, at least 100 mm, at least about 150 mm, or at least about 200 mm, or at least about 250 mm, or at least about 300 mm, or even at least about 500 mm. According to still other embodiments, the at least one retainer 120 may have an inner radius IR_{R} that may be not greater than about 1500 mm, not greater than about 1200 mm, or even not greater than about 1000 mm. It will be appreciated that the at least one retainer 120 may have an inner radius IR_{R} that may be within a range between any of the minimum and maximum values noted above. It will be further appreciated that the at least one retainer 120 may have an inner radius IR_{R} that may be any value between any of the minimum and maximum values noted above.

In a number of embodiments, as shown best in FIG. 1F2, the at least one retainer 120 can have an axial length (or thickness) L_{R}. For purposes of embodiments described herein, the length L_{R} of the at least one retainer 120 is the distance from the first axial end 112A to the second axial end 112B. According to certain embodiment, the length L_{R} of the at least one retainer 120 may be at least about 10 mm, at least 25 mm, at least 50 mm, at least 100 mm, at least about 150 mm, or at least about 200 mm, or at least about 250 mm, or at least about 300 mm, or even at least about 500 mm. According to still other embodiments, the length L_{R} of the at least one retainer 120 may be not greater than about 1500 mm, not greater than about 1200 mm, or even not greater than about 1000 mm. It will be appreciated that the length L_{R} of the at least one retainer 120 may be within a range between any of the minimum and maximum values noted above. It will be further appreciated that the length L_{R} of the at least one retainer 120 may be any value between any of the minimum and maximum values noted above.

In a number of embodiments, as shown best in FIG. 1F1, the at least one retainer 120 may include at least one axially oriented coupling component 124. In a number of embodiments, the at least one axially oriented coupling component 124 of the at least one retainer 120 may couple to a core 110 within the apparatus. In a number of embodiments, the at least one axially oriented coupling component 124 of the at least one retainer 120 may couple to a neighboring retainer 120 of a neighboring apparatus 100. In a number of embodiments, as shown best in FIG. 1F2, the at least one retainer 120 may include a plurality of axially oriented coupling components 124A, 124B. In a number of embodiments, the at least one axially oriented coupling component 124 may include at least one of nuts, bolts, bearings, battens, buckles, clips, flanges, frogs, grommets, hook-and-eyes, latches, pegs, nails, rivets, tongue and grooves, screw anchors, snap fasteners, stitches, threaded fasteners, ties, toggle bolts, wedges anchors, screws, bolts, clamps, clasps, clips, latches, pins, rivets, ties, nails, projection, bores, welds, or combinations thereof. In particular embodiments, as shown in FIG. 1F2, the at least one axially oriented coupling component 124 may include a tongue adapted to pair with a groove or bore of the core 100 to snap-fit the core and the retainer together. In particular embodiments, as shown in FIG. 1F1, the at least one axially oriented coupling component 124 may include a projection or groove to pair the at least one retainer 120 to a neighboring membrane 130 of a neighboring apparatus 100.

FIG. 1G1 includes a top view of a membrane for an apparatus in accordance with an embodiment. FIG. 1G2 includes an isometric view of a membrane for an apparatus in accordance with an embodiment. As shown in FIGS. 1G1-1G2, the at least one membrane 130 may include an annular body 132 forming an “annular shape” to be placed over the internal void 150 corresponding to the internal void of the apparatus 100 down a central axis 1000 to form a medically, biologically, or pharmaceutically active environment. In particular embodiments, the at least one membrane 130 may form a substantially circular shape with an outer radial end 132D, a first axial end 132A and a second axial end 132B, as shown, and defining a major surface 136. In an embodiment, the at least one membrane 130 can include a media or particulate for filtering. In another embodiment, the at least one membrane 130 can include a porous structure such as a block or structure of material having perforations or micro-perforations. In a further embodiment, the at least one membrane 130 can include a mesh, a screen, or a woven or non-woven medium. In yet another embodiment, the at least one membrane 130 can include a combination of filter elements. In a number of embodiments, the at least one membrane 130 may include a polymer that is gas-permeable and liquid-impermeable. In a number of embodiments, the at least one membrane 130 may include a polymer including ethylene-vinyl acetate (EVA), nitrile butadiene rubber (NBR), high density polyethylene (HDPE), silicone, polyolefin, polycarbonate, thermoplastic polyolefin elastomer (POE), cyclic olefin copolymer (COC), cyclo olefin polymer (COP), polymethylpentene (PMP), fluorinated ethylene propylene (FEP), polytetrafluoroethylene (PTFE), textured films (e.g. textured silicone, textured FEP, textured laminate), or a combination thereof. In a number of embodiments, the at least one membrane 130 may be a monolayer. In a number of embodiments, the at least one membrane 130 may be a multi-layer laminate.

In a number of embodiments, as shown best in FIG. 1G1, the at least one membrane 130 may have a radius OR_{M}. For purposes of embodiments described herein, the at least one membrane 130 may have an outer radius OR_{M} that is the distance from the central axis 1000 to the outer radial end 132D. According to certain embodiments, the at least one membrane 130 may have an outer radius OR_{M} that may be at least about 10 mm, at least 25 mm, at least 50 mm, at least 100 mm, at least about 150 mm, or at least about 200 mm, or at least about 250 mm, or at least about 300 mm, or even at least about 500 mm. According to still other embodiments, the at least one membrane 130 may have an outer radius OR_{M} that may be not greater than about 1500 mm, not greater than about 1200 mm, or even not greater than about 1000 mm. It will be appreciated that the at least one membrane 130 may have an outer radius OR_{M} that may be within a range between any of the minimum and maximum values noted above. It will be further appreciated that the at least one membrane 130 may have an outer radius OR_{M} that may be any value between any of the minimum and maximum values noted above.

In a number of embodiments, as shown best in FIG. 1G2, the at least one membrane 130 can have an axial length (or thickness) L_{M}. For purposes of embodiments described herein, the length L_{M} of the at least one membrane 130 is the distance from the first axial end 132A to the second axial end 132B. According to certain embodiment, the length L_{M} of the at least one membrane 130 may be at least about 0.0001 mm, at least 0.001 mm, at least 0.01 mm, at least 1 mm, at least 10 mm, at least 25 mm, at least 50 mm, at least 100 mm, at least about 150 mm, or at least about 200 mm, or at least about 250 mm, or at least about 300 mm, or even at least about 500 mm. According to still other embodiments, the length L_{M} of the at least one membrane 130 may be not greater than about 1500 mm, not greater than about 1200 mm, or even not greater than about 1000 mm. It will be appreciated that the length L_{M} of the at least one membrane 130 may be within a range between any of the minimum and maximum values noted above. It will be further appreciated that the length L_{M} of the at least one membrane 130 may be any value between any of the minimum and maximum values noted above.

In a number of embodiments, as shown best in FIG. 1G1, the at least one membrane 130 may include at least one axially oriented coupling component 134. In a number of embodiments, the at least one axially oriented coupling component 134 of the at least one membrane 130 may couple to a core 110 and/or retainer 120 within the apparatus. In a number of embodiments, as shown best in FIG. 1G2, the at least one membrane 130 may include a plurality of axially oriented coupling components 134A, 134B. In a number of embodiments, the at least one axially oriented coupling component 134 may include at least one of nuts, bolts, bearings, battens, buckles, clips, flanges, frogs, grommets, hook-and-eyes, latches, pegs, nails, rivets, tongue and grooves, screw anchors, snap fasteners, stitches, threaded fasteners, ties, toggle bolts, wedges anchors, screws, bolts, clamps, clasps, clips, latches, pins, rivets, ties, nails, projection, bores, welds, or combinations thereof. In particular embodiments, as shown in FIG. 1G2, the at least one axially oriented coupling component 134 may include a bore adapted to pair with a groove or bore of the core 100 to snap-fit the core and the membrane together with the membrane contained. In a further embodiment, the at least one membrane 130 can be adhered to at least one of the retainer 120 or the core 110 by an adhesive or compound. In a number of embodiments, referring back to FIGS. 1C-1D, the at least one retainer 120 may be adapted to couple to the top axial surface 111 or the bottom axial surface 113 of the annular body 112 of the core 110 to form a circumferential engagement cavity 140 which may be formed and defined radially between the at least one retainer 120 and the core 110 such that the core 110 forms the outer diameter of the at least one circumferential engagement cavity 140 while the at least one retainer 120 forms the inner diameter of the at least one circumferential engagement cavity 140 relative to the central axis 1000. In a number of embodiments, as shown, the apparatus can generally include at least one ring seal 160 and at least a part of a membrane 130 disposed within the engagement cavity 140. As shown in FIGS. 1C-1D, the unique architecture of the circumferential engagement cavity 140 may allow the membrane 130 to be fixed through the interaction of the at least one retainer 120, the core 110, and the ring seal 160 within the engagement cavity 140 to at least partially enclose the internal void 150. As shown in FIGS. 1C-1D, the unique architecture of the circumferential engagement cavity 140 may allow the membrane 130 to be fixed in a uniform circumferential pattern through the interaction of the at least one retainer 120, the core 110, and the ring seal 160 within the engagement cavity 140 to at least partially enclose the internal void 150.

In a number of embodiments, the at least one membrane 130 may be fixed within an engagement cavity 140 such that it has a flatness against a line or plane perpendicular to the central axis 1000 of the apparatus. In a number of embodiments, the at least one membrane 130 may be fixed within an engagement cavity 140 to have a deviation of at least about 0.0001 mm, at least 0.001 mm, at least 0.01 mm, at least 1 mm, at least 10 mm, at least 25 mm, at least 50 mm, at least 100 mm, at least about 150 mm, or at least about 200 mm, or at least about 250 mm, or at least about 300 mm, or even at least about 500 mm from a line or plane perpendicular to the central axis 1000 of the apparatus. According to still other embodiments, the at least one membrane 130 may be fixed within an engagement cavity 140 to have a deviation that may be not greater than about 1500 mm, not greater than about 1200 mm, or even not greater than about 1000 mm a line or plane perpendicular to the central axis 1000 of the apparatus. It will be appreciated that the at least one membrane 130 may be fixed within an engagement cavity 140 to have a deviation that may be within a range between any of the minimum and maximum values noted above. It will be further appreciated that the at least one membrane 130 may be fixed within an engagement cavity 140 to have a deviation that may be any value between any of the minimum and maximum values noted above.

FIG. 1H1 includes an isometric view of a retainer for an apparatus in accordance with an embodiment. FIG. 1H2 includes an isometric view of a retainer and a membrane for an apparatus in accordance with an embodiment. FIG. 1H3 includes an isometric cross-sectional view of a retainer and a membrane for an apparatus in accordance with an embodiment. In a number of embodiments, the at least one retainer 120 may be expected to have the same features as retainers otherwise described herein. As shown in FIGS. 1H1-1H3, in a number of alternative embodiments, the at least one retainer 120 may include a body 122. However, as shown in FIGS. 1H1-1H3, in a number of embodiments, the at least one retainer 120 may have a body 122 that includes an inner portion 122C and an outer portion 122D. In a number of embodiments, the body 122 (including at least one of the inner portion 122C or outer portion 122D) may be made of a rigid material as described below. In a number of embodiments, the body 122 (including at least one of the inner portion 122C or outer portion 122D) may be made of a rigid material including a rigid polymer as described below. In a number of embodiments, the body 122 (including at least one of the inner portion 122C or outer portion 122D) may be made of a rigid material including a rigid polymer including a polycarbonate.

In a number of embodiments, as shown in FIG. 1H1, the body 122 of the at least one retainer 120 may include coupling components 124 as described elsewhere herein. These coupling components 124 may be located on the outer portion 122D of the body 122 of the at least one retainer 120. In a number of embodiments, the body 122 of the at least one retainer may include an inner portion 122C with at least one through hole 126. In a number of embodiments, the body 122 of the at least one retainer may include an inner portion 122C with a plurality of through holes 126. In a number of embodiments, the at least one through hole 126 may have an arcuate, rectilinear, circular, polygonal, or oval cross-section, or a combination thereof. In a number of embodiments, at least one through hole 126 may have a major length (e.g. diameter) perpendicular to the central axis 1000 that varies in the axial direction to form an axial step in the through hole 126. The axial step may occur at any point along the axial length of the at least one through hole 126.

In a number of embodiments, the at least one through hole 126 may provide the inner portion 122C of the body 122 of the at least one retainer with a void area of at least 1%, at least 5%, at least 10%, at least 15%, at least 20%, at least 25%, at least 30%, at least 40%, at least 45%, at least 50%, at least 55%, or at least 60%. In a number of embodiments, the through holes 126 may provide the inner portion 122C of the body 122 of the at least one retainer with a void area of no greater than 95%, no greater than 90%, no greater than 85%, no greater than 80%, no greater than 75%, no greater than 70%, no greater than 65%, or no greater than 60%. In a number of embodiments, the void area may be in the range of 3.5% to 80% of a surface area of the rigid inner portion 122C of the at least one retainer 120. It will be appreciated that the void area may be within a range between any of the minimum and maximum values noted above. It will be further appreciated that the void area may be any value between any of the minimum and maximum values noted above

In a number of embodiments, as shown in FIGS. 1H1-1H3, in a number of alternative embodiments, the at least one retainer 120 may be bonded to the at least one flexible film membrane 130. In a number of embodiments, the at least one flexible film membrane 130 may be expected to have the same features as membranes otherwise described herein. In a number of embodiments, similar to other embodiments, the at least one retainer 120 can include a plurality of retainers 120A, 120B. Further, the at least one membrane 130 can include a plurality of membranes 130A, 130B which may together at least partially enclose the internal void 150 completely in the axial direction down the central axis 1000 to provide for a medically, biologically, or pharmaceutically active environment within the internal void 150 of the core 110 and subsequently the apparatus 100 as described herein. In a number of embodiments, the plurality of flexible film membranes includes a first flexible film membrane 120A partially disposed on the top axial surface 111 of the core 110 and a second flexible film membrane 120B partially disposed on the bottom axial surface 113 of the core 110 as described herein.

As shown in FIGS. 1H2-1H3, the at least one retainer 120 may be bonded to the at least one flexible film membrane 130 via lamination, overmolding, physical adhesion, chemical adhesion, or a combination thereof. In this way, the at least one flexible film membrane 130 may have a peel strength of at least 0.5 N/in, at least 1 N/in, at least 2 N/in, at least 3 N/in, at least 4 N/in, at least 5 N/in, at least 6 N/in, at least 7 N/in, at least 10 N/in, at least 15 N/in, or at least 20 N/in with the at least one retainer 120. It will be appreciated that the peel strength may be within a range between any of the minimum and maximum values noted above. It will be further appreciated that the peel strength may be any value between any of the minimum and maximum values noted above. Further, in this way, the at least one flexible film membrane 130 may be fixed by the bond between the at least one flexible film membrane 130 and the at least one retainer 120 such that it has a flatness having a deviation of no greater than 3 mm against a line perpendicular to a central axis 1000. In a number of embodiments, the at least one membrane 130 may be bonded to the at least one retainer 120 along an axial face 125 of the rigid inner portion 122C of the at least one retainer 120. In a number of embodiments, the at least one membrane 130 may be bonded to the at least one retainer 120 along a sidewall 127 of the axial face 125 of the rigid inner portion 122C of the at least one retainer 120. In a number of embodiments, the at least one membrane 130 may be bonded to the at least one retainer 120 along a radial sidewall 129 of at least one through hole 126 of the rigid inner portion 122C of the at least one retainer 120. In this way, the at least one flexible film membrane 130 may be at least partially disposed within a through hole 126 of the rigid inner portion 122C of the at least one retainer 120.

FIG. 1H4 includes an isometric cross-sectional view of a membrane for an apparatus in accordance with an embodiment. Further, in a number of embodiments as shown in FIG. 1H4, the ring seal 160 of other apparatuses shown herein may be replaced by a gasket 162 contacting or uniform with the at least one membrane 130. The gasket may be made of the same material as the at least one membrane 130 or may be a different material as described below.

In a number of embodiments, the apparatus 100 or any components thereof listed herein (including, but not limited to, the core 110, the at least one retainer 120, the at least one membrane 130, the ring seal 160, or the gasket 162) can include a single-piece body. In another embodiment, the apparatus 100 or any components thereof listed herein (including, but not limited to, the core 110, the at least one retainer 120, the at least one membrane 130, the ring seal 160, or the gasket 162) can include a multi-piece construction. In a number of embodiments, the apparatus 100 or any components thereof listed herein (including, but not limited to, the core 110, the at least one retainer 120, the at least one membrane 130, the ring seal 160, or the gasket 162) can include a single use apparatus. In another embodiment, the apparatus 100 or any components thereof listed herein (including, but not limited to, the core 110, the at least one retainer 120, the at least one membrane 130, the ring seal 160, or the gasket 162) can be reusable.

The apparatus 100 or any components thereof listed herein (including, but not limited to, the core 110, the at least one retainer 120, the at least one membrane 130, the ring seal 160, or the gasket 162) can be formed from any suitable material in the sealing arts. In a particular embodiment, the apparatus 100 or any components thereof listed herein can at least partially include a polymer. The polymer may be a thermoplastic or thermosetting polymer. The polymer may be selected from the group including a polyketone, a polyaramid, a polyphenylene sulfide, a polyethersulfone, a polyphenylene sulfone, a polyamideimide, ultra high molecular weight polyethylene, a fluoropolymer, a polybenzimidazole, a polyacetal, polybutylene terephthalate (PBT), polypropylene (PP), rubber modified polypropylene, (EPDM/PP), polycarbonate (PC), Acrylonitrile butadiene styrene (ABS), polyethylene terephthalate (PET), a polyimide (PI), polyetherimide, polyetheretherketone (PEEK), polyatheretherketone (PAEK), polyethylene (PE), a polysulfone, a polyamide (PA), thermoplastic polyurethane (TPU), polyphenylene oxide, polyphenylene sulfide (PPS), a polyurethane, a polyester, a liquid crystal polymer (LCP), an elastomer, or any combination thereof. In an embodiment, the apparatus 100 or any components thereof listed herein may include, or even consist essentially of, a fluoropolymer. Exemplary fluoropolymers include a polytetrafluoroethylene (PTFE), a modified PTFE (TFM), a fluorinated ethylene propylene (FEP), a polyvinylidene fluoride (PVDF), a perfluoroalkoxy (PFA), a terpolymer of tetrafluoroethylene, a hexafluoropropylene and vinylidene fluoride (THV), a polychlorotrifluoroethylene (PCTFE), an ethylene tetrafluoroethylene copolymer (ETFE), an ethylene chlorotrifluoroethylene copolymer (ECTFE), or any combination thereof. Other fluoropolymers, polymers, and blends may be included in the composition of the apparatus 100 or any components thereof listed herein. In another particular embodiment, the apparatus 100 or any components thereof listed herein can at least partially include, or even consist essentially of, a polyethylene (PE) such as an ultra-high-molecular-weight polyethylene (UHMWPE). In a number of embodiments, the apparatus 100 or any components thereof listed herein (including, but not limited to, the core 110, the at least one retainer 120, the at least one membrane 130, the ring seal 160, or the gasket 162) may include a polymer including ethylene-vinyl acetate (EVA), nitrile butadiene rubber (NBR), high density polyethylene (HDPE), silicone, polyolefin, polycarbonate, thermoplastic polyolefin elastomer (POE), cyclic olefin copolymer (COC), cyclo olefin polymer (COP), polymethylpentene (PMP), fluorinated ethylene propylene (FEP), polytetrafluoroethylene (PTFE), textured films (e.g. textured silicone, textured FEP, textured laminate), or a combination thereof. In another particular embodiment, the apparatus 100 or any components thereof listed herein may include a thermoplastic elastomeric hydrocarbon block copolymer, a polyether-ester block co-polymer, a thermoplastic polyamide elastomer, a thermoplastic polyurethane elastomer, a thermoplastic polyolefin elastomer, a thermoplastic vulcanizate, an olefin-based co-polymer, an olefin-based ter-polymer, a polyolefin plastomer, or combinations thereof. In an embodiment, the apparatus 100 or any components thereof listed herein may include a styrene based block copolymer such as styrene-butadiene, styrene-isoprene, blends or mixtures thereof, and the like. Exemplary styrenic thermoplastic elastomers include triblock styrenic block copolymers (SBC) such as styrene-butadiene-styrene (SBS), styrene-isoprene-styrene (SIS), styrene-ethylene butylene-styrene (SEBS), styrene-ethylene propylene-styrene (SEPS), styrene-ethylene-ethylene-butadiene-styrene (SEEBS), styrene-ethylene-ethylene-propylene-styrene (SEEPS), styrene-isoprene-butadiene-styrene (SIBS), or combinations thereof. Commercial examples include some grades of Kraton^{TM} and Hybrar^{TM} resins. In an embodiment, the apparatus 100 or any components thereof listed herein may include an elastomer including at least one of Acrylonitrile-Butadiene (NBR) Carboxylated Nitrile (XNBR) Ethylene Acrylate (AEM, Vamac®), Ethylene Propylene Rubber (EPR, EPDM), Butyl Rubber (IIR), Chloroprene Rubber (CR), Fluorocarbon (FKM, FPM), Fluorosilicone (FVMQ), Hydrogenated Nitrile (HNBR), Perfluoroelastomer (FFKM), Polyacrylate (ACM), Polyurethane (AU, EU), Silicone Rubber (Q, MQ, VMQ, PVMQ), Tetrafluoroethylene-Propylene (AFLAS®) (FEPM). In a number of embodiments, the apparatus 100 or any components thereof listed herein may be formed from any conventional methods known for polymer manufacturing, including but not limited to injection molding, billeting, cutting/slicing, hot or cold pressing, direct forming, extrusion, skiving, machining, blow molding, overmolding, vacuum forming, or CNC machining.

In an embodiment, the apparatus 100 or any components thereof listed herein (including, but not limited to, the core 110, the at least one retainer 120, the at least one membrane 130, the ring seal 160, or the gasket 162) can at least partially include a rigid material such as, but not limited to, a metal or any of the polymers listed above. According to certain embodiments, the metal may include iron, copper, titanium, tin, aluminum, alloys thereof, or may be another type of metal. In an embodiment, the apparatus 100 or any components thereof listed herein can include a metal (such as aluminum, zinc, copper, magnesium, tin, platinum, titanium, tungsten, iron, bronze, steel, energizer steel, stainless steel), a metal alloy (including the metals listed), an anodized metal (including the metals listed), or any combination thereof. In a number of embodiments, the apparatus 100 or any components thereof listed herein may be formed from any conventional methods known for metalworking, such as turning, forging, extruding, molding, micromolding, micromachining, sintering, rolling, casting, injection molding, metalworking, flattening, hydroforming, punching, CNC machining, or 3-D printing (including binderjetting, stereolithography, filament disposition method, laser melting, laser sintering, electron beam melting).

In an embodiment, the apparatus 100 or any components thereof listed herein (including, but not limited to, the core 110, the at least one retainer 120, the at least one membrane 130, the ring seal 160, or the gasket 162) can include a ceramic such as alumina, silica, titanium dioxide, calcium fluoride, boron nitride, mica, wollastonite, glass fibers, silicon carbide, silicon nitride, zirconia, carbon black, pigments, or any combination thereof. In a number of embodiments, the apparatus 100 or any components thereof listed herein may be formed from any conventional methods known for ceramic manufacturing, such as turning, extruding, molding, micromolding, micromachining, sintering, casting, injection molding, flattening, hydroforming, punching, CNC machining, or 3-D printing (including binderjetting, stereolithography, filament disposition method, laser melting, laser sintering, electron beam melting).

In an embodiment, the apparatus 100 or any components thereof listed herein (including, but not limited to, the core 110, the at least one retainer 120, the at least one membrane 130, the ring seal 160, or the gasket 162) can be treated, impregnated, filled, or coated with a lubricious material or filler as a coating or surface treatment. Exemplary lubricious materials or fillers include molybdenum disulfide, tungsten disulfide, graphite, grapheme, expanded graphite, boron nitride, talc, calcium fluoride, or any combination thereof. Additionally, the lubricious material or filler can include a ceramic such as alumina, silica, titanium dioxide, calcium fluoride, boron nitride, mica, wollastonite, glass fibers, silicon carbide, silicon nitride, zirconia, carbon black, pigments, or any combination thereof. In an embodiment, the apparatus 100 or any components thereof listed herein may be a material that is resistant to oxygen, hydrogen, hydroxide, or temperature resistant. In a number of embodiments, the apparatus 100 or any components thereof listed herein may be monolithic. In alternative embodiments, the apparatus 100 or any components thereof listed herein may not be monolithic and may include multiple pieces.

In an embodiment, the apparatus 100 or any components thereof listed herein (including, but not limited to, the core 110, the at least one retainer 120, the at least one membrane 130, the ring seal 160, or the gasket 162) can advantageously withstand sterilization processes. In an embodiment, the apparatus 100 or any components thereof listed herein may be sterilized by any method envisioned. For instance, the polymer of the apparatus 100 is sterilized after the apparatus 100 is formed. Exemplary sterilization methods include radiation (such as X-ray radiation), electron ray, E-beam or electron beam sterilization techniques, combinations thereof, and the like. In a particular embodiment, the polymer or polymeric blend is sterilized by vaporized hydrogen peroxide sterilization (VHP). In a particular embodiment, the apparatus 100 or any components thereof listed herein is sterilized by gamma irradiation. For instance, the apparatus 100 or any components thereof listed herein may be gamma sterilized at between about 25 kGy to about 50 kGy.

In embodiment, the apparatus 100 or any components thereof listed herein (including, but not limited to, the core 110, the at least one retainer 120, the at least one membrane 130, the ring seal 160, or the gasket 162) may have further desirable physical and mechanical properties. For instance, the apparatus 100 or any components thereof listed herein may appear transparent or at least translucent. For instance, the apparatus 100 or any components thereof listed herein may have a light transmission greater than about 2%, or greater than about 5% in the visible light wavelength range. In particular, the resulting articles have desirable clarity or translucency. In addition, the apparatus 100 or any components thereof listed herein may have advantageous physical properties, such as a balance of any one or more of the properties of hardness, flexibility, surface lubricity, tensile strength, elongation, Shore A hardness, gamma resistance, weld strength, and seal integrity to an optimum level.

In an embodiment, the apparatus 100 or any components thereof listed herein (including, but not limited to, the core 110, the at least one retainer 120, the at least one membrane 130, the ring seal 160, or the gasket 162) may have desirable heat stability properties. Applications for the polymer or polymeric blend are numerous. In particular, the apparatus 100 or any components thereof listed herein may be non-toxic, making the material useful for any application where no toxicity is desired. For example, the apparatus 100 or any components thereof listed herein may be substantially free of plasticizers or other low-molecular weight extenders that can be leached into the fluids it transfers. “Substantially free” as used herein refers to a polymeric mixture having a total organic content (TOC) (measured in accordance to ISO 15705 and EPA 410.4) of less than about 100 ppm. Further, the apparatus 100 or any components thereof listed herein may have biocompatibility and animal derived component-free formulation ingredients. For instance, the apparatus 100 or any components thereof listed herein may have potential for FDA, USP, EP, ISO, and other regulatory approvals. In an exemplary embodiment, the apparatus 100 or any components thereof listed herein may be used in applications such as industrial, medical, health care, biopharmaceutical, pharmaceutical, drinking water, food & beverage, laboratory, dairy, and the like. In an embodiment, the apparatus 100 or any components thereof listed herein may be used in applications where low temperature resistance is desired. In an embodiment, the apparatus 100 or any components thereof listed herein may also be safely disposed as it generates substantially no toxic gases when incinerated and leaches no plasticizers into the environment if land filled.

In some embodiments, as shown best in FIG. 1D, the apparatus 100 may be configured to contain and modify and/or sustain a product 170 within its internal void 150 to form a system. The product 170 may include medium containing a medical, biological, or pharmaceutical component. In a number of embodiments, the medical, biological, or pharmaceutical component may at least one of a cell, biofluid, virus, microphage, protein, antibody, pharmaceutical, therapeutic, buffer, or combinations thereof. In a number of embodiments, the product 170 may include a fluid for use in medical, biological, or pharmaceutical applications. In a number of embodiments, the product 170 may include a biomedia fluid for use in medical, biological, or pharmaceutical applications such as, but not limited to, cell cultures. In a number of embodiments, the medically, biologically, or pharmaceutically active environment within the apparatus 100 according to embodiments herein may be adapted to undergo a biological process or chemical reaction to modify or sustain the medical, biological, or pharmaceutical component 170 within the medium as provided by the medically, biologically, or pharmaceutically active environment within the internal void 150 of the apparatus 100.

In accordance with embodiments described herein, a method of making a system for storing medical, pharmaceutical, or biological media is shown. The method can include providing a medium including a medical, pharmaceutical, or biological component 170. The method can further include providing an apparatus 100 including: a core 110 including an annular body 112 defining an internal void 150 oriented down a central axis 1000 having a top axial surface 112A and a bottom axial surface 112B, at least one retainer 120 including an annular body 122 adapted to couple to the top axial surface 112A or the bottom axial surface 112B of the core 100 to form a circumferential engagement cavity 140 defined radially between the at least one retainer 120 and the core 110, at least one ring seal 160 disposed within the engagement cavity 140, and at least one flexible film membrane 130 partially disposed within the engagement cavity 140, where the at least one flexible film membrane 130 at least partially encloses the internal void 150. The method may further include disposing the medium 170 within the internal void 150 to provide for a medically, biologically, or pharmaceutically active environment within the internal void 150 of the core 110 and subsequent apparatus 100.

In accordance with embodiments described herein, a method for using a system is shown. The method may include providing a medium 170 including a medical, pharmaceutical, or biological component. The method may further include providing an apparatus 100 including: a core 110 including an annular body 112 defining an internal void 150 oriented down a central axis 1000 having a top axial surface 112A and a bottom axial surface 112B, at least one retainer 120 including an annular body 122 adapted to couple to the top axial surface 112A or the bottom axial surface 112B of the core 110, where the at least one retainer 120 includes a rigid inner portion 122C including a plurality of through holes 126, and at least one flexible film membrane 130 bonded to the at least one retainer 120, where the at least one flexible film membrane 130 at least partially encloses the internal void 150. The method may further include disposing the medium 170 within the internal void 150 to provide for a medically, biologically, or pharmaceutically active environment within the internal void 150 of the core 110. The method may further include removing the medium 170 from the internal void 150, where at least one of disposing the medium 170 within the internal void 150 or removing the medium 170 from the internal void 150 maintains the at least one film membrane 130 in a substantially static axial position via the at least one retainer 120.

In a particular instance, apparatuses, systems and methods described herein can be adapted for use with medical, biological, or pharmaceutical media. In an embodiment, the apparatuses, systems and methods described herein can be used to prevent wrinkling, enabling more efficient storage, better gas diffusion, better handling, and better operation involving modification or sustainment of the media within the apparatus unlike previous or conventional apparatuses of similar use. This may lengthen the lifetime and efficiency of the apparatus and the medical, biological, or pharmaceutical media cultivated therein.

Many different aspects and embodiments are possible. Some of those aspects and embodiments are described below. After reading this specification, skilled artisans will appreciate that those aspects and embodiments are only illustrative and do not limit the scope of the present invention. Embodiments may be in accordance with any one or more of the items as listed below.

Embodiment 1: An apparatus comprising: a core comprising an annular body defining an internal void oriented down a central axis having a top axial surface and a bottom axial surface; at least one retainer comprising a body adapted to couple to the top axial surface or the bottom axial surface of the core; and at least one flexible film membrane bonded to the at least one retainer, wherein the at least one flexible film membrane at least partially encloses the internal void to provide for a medically, biologically, or pharmaceutically active environment within the internal void of the core, wherein the at least one retainer comprises a rigid inner portion comprising a plurality of through holes, wherein the at least one retainer is adapted to hold the at least one flexible film membrane in a substantially static axial position.

Embodiment 2: A system comprising: a medium comprising a medical, pharmaceutical, or biological component; and an apparatus comprising: a core comprising an annular body defining an internal void oriented down a central axis having a top axial surface and a bottom axial surface, at least one retainer comprising a body adapted to couple to the top axial surface or the bottom axial surface of the core, and at least one flexible film membrane bonded to the at least one retainer, wherein the at least one flexible film membrane at least partially encloses the internal void about the medium to provide for a medically, biologically, or pharmaceutically active environment within the internal void of the core, wherein the at least one retainer comprises a rigid inner portion comprising a plurality of through holes, wherein the at least one retainer is adapted to hold the at least one flexible film membrane in a substantially static axial position.

Embodiment 3: A method comprising: providing a medium comprising a medical, pharmaceutical, or biological component; providing an apparatus comprising: a core comprising an annular body defining an internal void oriented down a central axis having a top axial surface and a bottom axial surface, at least one retainer comprising an annular body adapted to couple to the top axial surface or the bottom axial surface of the core, wherein the at least one retainer comprises a rigid inner portion comprising a plurality of through holes, and at least one flexible film membrane bonded to the at least one retainer, wherein the at least one flexible film membrane at least partially encloses the internal void; disposing the medium within the internal void to provide for a medically, biologically, or pharmaceutically active environment within the internal void of the core; and removing the medium from the internal void, wherein at least one of disposing the medium within the internal void or removing the medium from the internal void maintains the at least one film membrane in a substantially static axial position via the at least one retainer.

Embodiment 4: The apparatus, system, or method of any of the preceding embodiments, wherein the at least one retainer comprises a plurality of retainers, wherein a first retainer couples to the top axial surface of the core and a second retainer couples to a bottom axial surface of the core.

Embodiment 5: The apparatus, system, or method of any of the preceding embodiments, wherein the at least one flexible film membrane comprises a plurality of flexible film membranes.

Embodiment 6: The apparatus, system, or method of any of the preceding embodiments, wherein the plurality of flexible film membranes comprises a first flexible film membrane at least partially disposed on the top axial surface of the core and a second flexible film membrane at least partially disposed on the bottom axial surface of the core.

Embodiment 7: The apparatus, system, or method of any of the preceding embodiments, wherein the at least one flexible film membrane comprises a polymer that is gas-permeable and liquid-impermeable.

Embodiment 8: The apparatus, system, or method of embodiment 7, wherein the polymer comprises ethylene-vinyl acetate (EVA), nitrile butadiene rubber (NBR), high density polyethylene (HDPE), silicone, polyolefin, polycarbonate, thermoplastic polyolefin elastomer (POE), cyclic olefin copolymer (COC), cyclo olefin polymer (COP), polymethylpentene (PMP), fluorinated ethylene propylene (FEP), polytetrafluoroethylene (PTFE), textured films (e.g. textured silicone, textured FEP, textured laminate), or a combination thereof.

Embodiment 9: The apparatus, system, or method of embodiment 7, wherein the polymer comprises silicone.

Embodiment 10: The apparatus, system, or method of any of the preceding embodiments, wherein the at least one flexible film membrane comprises a monolayer.

Embodiment 11: The apparatus, system, or method of any of the preceding embodiments, wherein the at least one flexible film membrane comprises a multilayer laminate.

Embodiment 12: The apparatus, system, or method of any of the preceding embodiments, wherein the at least one flexible film membrane has a thickness of greater than 0.001 inches and less than 0.01 inches.

Embodiment 13: The apparatus, system, or method of any of the preceding embodiments, wherein the plurality of through holes provide the rigid inner portion of the at least one retainer with a void area in the range of 3.5% to 80% of a surface area of the rigid inner portion of the at least one retainer.

Embodiment 14: The apparatus, system, or method of any of the preceding embodiments, wherein at least one through hole of the plurality of through holes has an arcuate, rectilinear, circular, polygonal, or oval cross-section, or a combination thereof.

Embodiment 15: The apparatus, system, or method of any of the preceding embodiments, wherein at least one through hole of the plurality of through holes has a major length that varies in the axial direction.

Embodiment 16: The apparatus, system, or method of any of the preceding embodiments, wherein the at least one flexible film membrane is at least partially disposed within a through hole of the rigid inner portion of the at least one retainer.

Embodiment 17: The apparatus, system, or method of any of the preceding embodiments, wherein the at least one flexible film membrane is fixed by the bond between the at least one flexible film membrane and the at least one retainer such that it has a flatness having a deviation of no greater than 3 mm against a line perpendicular to a central axis.

Embodiment 18: The apparatus, system, or method of any of the preceding embodiments, wherein the at least one flexible film membrane has a peel strength of 1 N/in with the at least one retainer.

Embodiment 19: The apparatus, system, or method of any of the preceding embodiments, wherein the at least one membrane is bonded to the at least one retainer along a radial sidewall of at least one through hole of the rigid inner portion of the at least one retainer.

Embodiment 20: The apparatus, system, or method of any of the preceding embodiments, wherein the at least one membrane is bonded to the at least one retainer along an axial face of the rigid inner portion of the at least one retainer.

Embodiment 21: The apparatus, system, or method of any of the preceding embodiments, wherein the rigid inner portion of the at least one retainer comprises an axial face having a sidewall, wherein the at least one membrane is bonded to the at least one retainer along the sidewall of the axial face of the rigid inner portion of the at least one retainer.

Embodiment 22: The apparatus, system, or method of any of the preceding embodiments, wherein the bonding between the at least one membrane to the at least one retainer is done via lamination, overmolding, injection molding, physical adhesion, chemical adhesion, or a combination thereof.

Embodiment 23: The apparatus, system, or method of any of the preceding embodiments, wherein the core comprises at least one axially oriented coupling component to couple with the core comprising at least one of nuts, bolts, bearings, battens, buckles, clips, flanges, frogs, grommets, hook-and-eyes, pegs, nails, rivets, tongue and grooves, screw anchors, snap fasteners, stitches, threaded fasteners, ties, toggle bolts, wedges anchors, screws, clamps, clasps, latches, pins, bores, welds, or combinations thereof.

Embodiment 24: The apparatus, system, or method of any of the preceding embodiments, wherein the at least one retainer comprises at least one axially oriented coupling component to couple with the core comprising at least one of nuts, bolts, bearings, battens, buckles, clips, flanges, frogs, grommets, hook-and-eyes, pegs, nails, rivets, tongue and grooves, screw anchors, snap fasteners, stitches, threaded fasteners, ties, toggle bolts, wedges anchors, screws, clamps, clasps, latches, pins, bores, welds, or combinations thereof.

Embodiment 25: The apparatus, system, or method of embodiment 24, wherein the at least one coupling component of the at least one retainer couples to a neighboring retainer of a neighboring apparatus.

Embodiment 26: The apparatus, system, or method of any of the preceding embodiments, wherein the annular body of the core comprises at least one access port disposed on the core, wherein the at least one access port allows for entrance or exit of a medium into or out of the apparatus.

Embodiment 27: The apparatus, system, or method of any of the preceding embodiments, further comprising a gasket contacting or uniform with the at least one membrane.

Embodiment 28: The apparatus, system, or method of any of embodiment 27, wherein the gasket comprises a polymer comprising at least one ethylene-vinyl acetate (EVA), nitrile butadiene rubber (NBR), high density polyethylene (HDPE), silicone, polyolefin, polycarbonate, thermoplastic polyolefin elastomer (POE), cyclic olefin copolymer (COC), cyclo olefin polymer (COP), polymethylpentene (PMP), fluorinated ethylene propylene (FEP), polytetrafluoroethylene (PTFE), textured films (e.g. textured silicone, textured FEP, textured laminate), or a combination thereof.

Embodiment 29: The apparatus, system, or method of any of embodiment 27, wherein the gasket comprises a polymer comprising silicone.

Embodiment 30: The apparatus, system, or method of any of the preceding embodiments, wherein the at least one of the retainer comprises a rigid material comprising a ceramic.

Embodiment 31: The apparatus, system, or method of any of the preceding embodiments, wherein the at least one of the retainer comprises a rigid material comprising a polymer.

Embodiment 32: The apparatus, system, or method of any of the preceding embodiments, wherein the at least one of the retainer comprises a rigid material comprising a metal.

Embodiment 33: The apparatus, system, or method of any of the preceding embodiments, wherein the core comprises a rigid material comprising a ceramic.

Embodiment 34: The apparatus, system, or method of any of the preceding embodiments, wherein the core comprises a rigid material comprising a polymer.

Embodiment 35: The apparatus, system, or method of any of the preceding embodiments, wherein the core comprises a rigid material comprising a metal.

Embodiment 36: The apparatus, system, or method of any of the preceding embodiments, wherein the core has a substantially U-shaped cross-sectional profile in a plane parallel to the central axis.

Embodiment 37: The apparatus, system, or method of any of the preceding embodiments, wherein the at least one retainer has a modified L-shaped cross-sectional profile in a plane parallel to the central axis.

Embodiment 38: The apparatus, system, or method of any of the preceding embodiments, wherein the medically, biologically, or pharmaceutically active environment allows for a biological process to take place to modify or sustain the medium.

Embodiment 39: The apparatus, system, or method of any of the preceding embodiments, wherein the medical, pharmaceutical, or biological component comprises at least one of a cell, biofluid, virus, microphage, protein, antibody, pharmaceutical, therapeutic, or combinations thereof.

Embodiment 40: The apparatus, system, or method of any of the preceding embodiments, wherein the apparatus comprises a surface treatment or coating comprising a lubricious metal or a filler.

Note that not all of the activities described above in the general description or the examples are required, that a portion of a specific activity may not be required, and that one or more further activities may be performed in addition to those described. Still further, the order in which activities are listed is not necessarily the order in which they are performed.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Separate embodiments may also be provided in combination in a single embodiment, and conversely, various features that are, for brevity, described in the context of a single embodiment, may also be provided separately or in any subcombination. Further, reference to values stated in ranges includes each and every value within that range. Many other embodiments may be apparent to skilled artisans only after reading this specification. Other embodiments may be used and derived from the disclosure, such that a structural substitution, logical substitution, or another change may be made without departing from the scope of the disclosure. Accordingly, the disclosure is to be regarded as illustrative rather than restrictive.

What is claimed is:

1. An apparatus comprising:
a core comprising an annular body defining an internal void oriented down a central axis having a top axial surface and a bottom axial surface;
at least one retainer comprising a body adapted to couple to the top axial surface or the bottom axial surface of the core; and
at least one flexible film membrane bonded to the at least one retainer, wherein the at least one flexible film membrane at least partially encloses the internal void to provide for a medically, biologically, or pharmaceutically active environment within the internal void of the core, wherein the at least one retainer comprises a rigid inner portion comprising a plurality of through holes, wherein the at least one retainer is adapted to hold the at least one flexible film membrane in a substantially static axial position.

2. A system comprising:
a medium comprising a medical, pharmaceutical, or biological component; and
an apparatus comprising:
a core comprising an annular body defining an internal void oriented down a central axis having a top axial surface and a bottom axial surface,
at least one retainer comprising a body adapted to couple to the top axial surface or the bottom axial surface of the core, and
at least one flexible film membrane bonded to the at least one retainer, wherein the at least one flexible film membrane at least partially encloses the internal void about the medium to provide for a medically, biologically, or pharmaceutically active environment within the internal void of the core, wherein the at least one retainer comprises a rigid inner portion comprising a plurality of through holes, wherein the at least one retainer is adapted to hold the at least one flexible film membrane in a substantially static axial position.

3. A method comprising:
providing a medium comprising a medical, pharmaceutical, or biological component;
providing an apparatus comprising:
a core comprising an annular body defining an internal void oriented down a central axis having a top axial surface and a bottom axial surface,
at least one retainer comprising an annular body adapted to couple to the top axial surface or the bottom axial surface of the core, wherein the at least one retainer comprises a rigid inner portion comprising a plurality of through holes, and
at least one flexible film membrane bonded to the at least one retainer, wherein the at least one flexible film membrane at least partially encloses the internal void;
disposing the medium within the internal void to provide for a medically, biologically, or
pharmaceutically active environment within the internal void of the core; and
removing the medium from the internal void, wherein at least one of disposing the medium within the internal void or removing the medium from the internal void maintains the at least one film membrane in a substantially static axial position via the at least one retainer.

4. The apparatus of claim 1, wherein the at least one retainer comprises a plurality of retainers, wherein a first retainer couples to the top axial surface of the core and a second retainer couples to a bottom axial surface of the core.

5. The apparatus of claim 1, wherein the at least one flexible film membrane comprises a plurality of flexible film membranes.

6. The apparatus of claim 1, wherein the plurality of flexible film membranes comprises a first flexible film membrane at least partially disposed on the top axial surface of the core and a second flexible film membrane at least partially disposed on the bottom axial surface of the core.

7. The apparatus of claim 1, wherein the at least one flexible film membrane comprises a polymer that is gas-permeable and liquid-impermeable.

8. The apparatus of claim 1, wherein the at least one flexible film membrane has a thickness of greater than 0.001 inches and less than 0.01 inches.

9. The apparatus of claim 1, wherein the plurality of through holes provide the rigid inner portion of the at least one retainer with a void area in the range of 3.5% to 80% of a surface area of the rigid inner portion of the at least one retainer.

10. The apparatus of claim 1, wherein at least one through hole of the plurality of through holes has an arcuate, rectilinear, circular, polygonal, or oval cross-section, or a combination thereof.

11. The apparatus of claim 1, wherein at least one through hole of the plurality of through holes has a major length that varies in the axial direction.

12. The apparatus of claim 1, wherein the at least one flexible film membrane is at least partially disposed within a through hole of the rigid inner portion of the at least one retainer.

13. The apparatus of claim 1, wherein the at least one flexible film membrane is fixed by the bond between the at least one flexible film membrane and the at least one retainer such that it has a flatness having a deviation of no greater than 3 mm against a line perpendicular to a central axis.

14. The apparatus of claim 1, wherein the at least one membrane is bonded to the at least one retainer along a radial sidewall of at least one through hole of the rigid inner portion of the at least one retainer.

15. The apparatus of claim 1, wherein the at least one membrane is bonded to the at least one retainer along an axial face of the rigid inner portion of the at least one retainer.

16. The apparatus of claim 1, wherein the rigid inner portion of the at least one retainer comprises an axial face having a sidewall, wherein the at least one membrane is bonded to the at least one retainer along the sidewall of the axial face of the rigid inner portion of the at least one retainer.

17. The apparatus of claim 1, wherein the core comprises at least one axially oriented coupling component to couple with the core comprising at least one of nuts, bolts, bearings, battens, buckles, clips, flanges, frogs, grommets, hook-and-eyes, pegs, nails, rivets, tongue and grooves, screw anchors, snap fasteners, stitches, threaded fasteners, ties, toggle bolts, wedges anchors, screws, clamps, clasps, latches, pins, bores, welds, or combinations thereof.

18. The apparatus of claim 1, wherein the at least one retainer comprises at least one axially oriented coupling component to couple with the core comprising at least one of nuts, bolts, bearings, battens, buckles, clips, flanges, frogs, grommets, hook-and-eyes, pegs, nails, rivets, tongue and grooves, screw anchors, snap fasteners, stitches, threaded fasteners, ties, toggle bolts, wedges anchors, screws, clamps, clasps, latches, pins, bores, welds, or combinations thereof.

19. The apparatus of claim 1, wherein the annular body of the core comprises at least one access port disposed on the core, wherein the at least one access port allows for entrance or exit of a medium into or out of the apparatus.

20. The apparatus of claim 1, further comprising a gasket contacting or uniform with the at least one membrane.
